(19) United States
(12) Patent Application Publication
(10) Pub. No.: US 2020/0323773 A1
(43) Pub. Date: Oct. 15, 2020

(54) FOOD AND BEVERAGE COMPOSITIONS COMPRISING PDE5 INHIBITORS

(71) Applicant: POVIVA CORP., Carson City, NV (US)

(72) Inventors: John Docherty, Port Perry (CA); Christopher Andrew Bunka, Kelowna (CA)

(21) Appl. No.: 16/761,920

(22) PCT Filed: Nov. 27, 2018
(86) PCT No.: PCT/US2018/062677

§ 371 Date: May 6, 2020

Related U.S. Application Data
(60) Provisional application No. 62/582,700, filed on Nov. 7, 2017.

(51) Int. Cl. A61K 9/00; A61K 47/36; A61K 47/44; A61K 47/46; A61K 31/355; A61K 31/352; A61K 31/05; A61K 31/519; A61K 31/506; A61K 31/4985; A61K 31/53; A61K 31/616; A61K 31/192; A61K 31/167; A61K 31/196; A61K 31/405; A61K 31/5415

(52) U.S. Cl. CPC A61K 9/0056; A61K 31/5415; A61K 47/44; A61K 47/46; A61K 31/355; A61K 31/352; A61K 31/05; A61K 31/519; A61K 31/506; A61K 31/4985; A61K 31/53; A61K 31/616; A61K 31/192; A61K 31/167; A61K 31/196; A61K 31/405; A61K 47/36

(57) ABSTRACT

Aspects described herein relate to improved compositions and methods for infusing food and beverage compositions with PDE5 inhibitors, alone or in combination with other lipophilic active agents such as cannabinoids, nicotine, non-steroidal anti-inflammatory drugs (NSAIDs), and vitamins.

### TECHNICAL FIELD

Aspects described herein relate to improved methods for infusing food and beverage compositions with PDE5 inhibitors, alone or in combination with other lipophilic active agents such as cannabinoids, nicotine, non-steroidal anti-inflammatory drugs (NSAIDs), and vitamins.

### BACKGROUND

Many therapeutic agents are highly lipophilic, meaning that they are soluble in lipids and some organic solvents while being substantially insoluble or only sparsely soluble in water. The poor water-solubility of these lipophilic agents often results in major difficulties in formulation. When administered in the form of an oil solution or some kind of water and/or oil suspension or emulsion, lipophilic compounds usually show poor bioavailability.

Various approaches to overcoming this limitation are known in the prior art. One such approach consists of dissolving a lipophilic compound in a water-miscible organic solvent such as ethanol or propylene glycol. However, when the resulting solution is admixed with blood or gastrointestinal fluids, the lipophilic compound usually precipitates as a solid or liquid emulsion, which results in a low bioavailability. Furthermore, for many lipophilic compounds no water-miscible organic solvents exist.

Another approach involves physico-chemical solubilization techniques such as micellar solubilization by means of surface-active agents (i.e., the use of surfactant micelles to solubilize and transport the therapeutic agent). In aqueous solution, micelles can incorporate lipophilic therapeutic agents in the hydrocarbon core of the micelle, or can entangle the agents at various positions within the micelle walls. Although micellar formulations can solubilize a variety of lipophilic therapeutic agents, the loading capacity of conventional micelle formulations is limited by the solubility of the therapeutic agent in the micelle surfactant. For many lipophilic therapeutic agents, such solubility is too low to offer formulations that can deliver therapeutically effective doses.

Another method consists of preparing a derivative or an analog of the lipophilic compound having a better solubility in water than the original compound. For example, this derivative may be a water-soluble salt of the compound that usually retains the original biological activity. However, this approach is applicable only to compounds having acidic or basic properties. If more substantial modifications are introduced into the original compound to improve its solubility, a decrease or even a complete loss of the original bioactivity of the compound is frequently observed.

Another approach consists of preparing a water-soluble pro-drug capable of liberating the original bioactive compound under physiological conditions. Such pro-drugs usually improve bioavailability of the compound and can ensure a targeted delivery of the compound or its sustained release over a period of time. However, the use of pro-drugs is not universally applicable since they usually require the presence of certain functional groups in the original compound. In addition, synthetic methods of improving solubility of a compound by chemical modifications are relatively complicated and expensive.

Other methods involve the formation of complexes by the addition of chelating agents such as citric acid, tartaric acid, amino acids, thioglycolic acid, and edetate disodium. Still other methods use buffering agents such as acetate, citrate, glutamate, and phosphate salts. However, buffers and chelating agents have been implicated in imparting high levels of aluminum in products, leading to adverse side effects. Furthermore, certain chelating agents such as EDTA have been implicated in adverse events such nephrotoxicity and renal tubular necrosis.

Therefore, there is a need for improved compositions and methods for the administration of lipophilic active agents to treat a variety of disorders in subjects in need thereof.

### SUMMARY

To address the foregoing problems, in whole or in part, and/or other problems that may have been observed by persons skilled in the art, the present disclosure provides compositions and methods as described by way of example as set forth below.

In one aspect, a lipophilic active agent infused food product is provided, comprising: (a) a therapeutically effective amount of a phosphodiesterase type 5 (PDE5 inhibitor), alone or in combination with one or more additional lipophilic active agents; (b) a bioavailability enhancing agent, wherein the bioavailability enhancing agent enhances the bioavailability of the lipophilic active agent(s); and (c) a food product. In another aspect, the lipophilic active agent infused food product is obtainable by the steps of: (i) contacting the food product with an oil comprising the PDE5 inhibitor, alone or in combination with a one or more additional lipophilic active agents, and the bioavailability enhancing agent; and (ii) dehydrating the food product; thereby producing the lipophilic active agent infused food product. In a further aspect, step (i) comprises saturating the food product in the oil comprising the PDE5 inhibitor, alone or in combination with a one or more additional lipophilic active agents, and the bioavailability enhancing agent. In another aspect, the lipophilic active agent infused food product further comprises: (d) a starch. In another aspect, step (ii) comprises contacting the food product with the starch.

In some aspects, the food product is selected from the group consisting of tea leaves, coffee beans, cocoa powder, meats, fish, fruits, vegetables, dairy products, legumes, pastas, breads, grains, seeds, nuts, spices, and herbs. In yet another aspect, the lipophilic active agent infused food product further comprises a flavoring agent, particularly wherein the flavoring agent is selected from the group consisting of vanilla, vanillin, ethyl vanillin, orange oil, peppermint oil, strawberry, raspberry, and mixtures thereof. In a further aspect, the lipophilic active agent infused food product is lyophilized. In a further aspect, wherein the lipophilic active agent infused food product is tea leaves, the process further comprises packaging the tea leaves in tea bags.

In another aspect, a lipophilic active agent infused beverage product is provided that is obtainable by the steps of: (i) providing lipophilic active agent infused tea leaves, coffee beans, or cocoa powder as described herein; and (ii) steeping the lipophilic active agent infused tea leaves, coffee beans, or cocoa powder in a liquid; thereby producing the lipophilic active agent infused beverage product.

In another aspect, a pharmaceutical composition is provided, comprising (a) a therapeutically effective amount of a PDE5 inhibitor, alone or in combination with one or more additional lipophilic active agents; and (b) a bioavailability enhancing agent, wherein the bioavailability enhancing agent enhances the bioavailability of the lipophilic active agent(s). In another aspect, the pharmaceutical composition further comprises: (c) a starch. In a further aspect, the pharmaceutical composition is formulated for oral administration. In some aspects, the pharmaceutical composition formulated for oral administration is formulated as a tablet, pill, capsule, liquid, gel, syrup, or slurry.

In some aspects, within the compositions and methods of the present invention, the PDE5 inhibitor is selected from the group consisting of avanafil, lodenafil, mirodenafil, sildenafil (or analogs thereof, for example, actetildenafil, hydroxyacetildenafil, or dimethyl-sildenafil), tadalafil, vardenafil, udenafil, acetildenafil, and thiome-thisosildenafil. In other aspects, the one or more additional lipophilic active agents is selected from the group consisting of a cannabinoid, nicotine, a non-steroidal anti-inflammatory drug (NSAID), and a vitamin. In other aspects, the cannabinoid is a nonpsychoactive cannabinoid such as cannabidiol. In other aspects, the NSAID is selected from the group consisting of acetylsalicylic acid, ibuprophen, acetaminophen, diclofenac, indomethacin, and piroxicam. In other aspects, the lipophilic active agent is vitamin E.

In some aspects, within the compositions and methods of the present invention, the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

In some aspects, within the compositions and methods of the present invention, the bioavailability enhancing agent is an edible oil or fat, a protective colloid, or both a protective colloid and an edible oil or fat. In another aspect, the bioavailability enhancing agent is also a lipophilic active agent taste masking agent. In another particular aspect, where the bioavailability enhancing agent is both a protective colloid, an edible oil or fat, and a lipophilic active agent taste masking agent, the bioavailability enhancing agent is nonfat dry milk. In a further aspect, the bioavailability enhancing agent is substantially free of omega-6 fatty acids. In other aspects, the bioavailability of the lipophilic active agent in a subject is at least about 1.5 times, 2 times, 5 times, or 10 times greater than the bioavailability of the lipophilic active agent in the subject in the absence of the bioavailability enhancing agent. In a further aspect, the bioavailability of the lipophilic active agent in a subject is greater than 20%.

In a further aspect, a method of treating one or more conditions is provided, comprising administering any of the compositions disclosed herein to a subject in need thereof. In one aspect, the condition is a PDE5-related disorder, particularly erectile dysfunction. In another aspect, where the one or more additional lipophilic active agents within the compositions and methods of the invention is a cannabinoid, the condition is selected from the group consisting of cardiac diseases such as heart disease, ischemic infarcts, and cardiometabolic disorders; neurological diseases such as Alzheimer's disease, Parkinson's disease, schizophrenia, and Human Immunodeficiency Virus (HIV) dementia; obesity; metabolic disorders such as insulin related deficiencies and lipid profiles, hepatic diseases, diabetes, and appetite disorders; cancer chemotherapy; benign prostatic hypertrophy; irritable bowel syndrome; biliary diseases; ovarian disorders; marijuana abuse; and alcohol, opioid, nicotine, or cocaine addiction. In another aspect, where the one or more additional lipophilic active agents within the compositions and methods of the invention is nicotine, the condition is a nicotine-related disorder. In another aspect, where the one or more additional lipophilic active agents within the compositions and methods of the invention is an NSAID as described herein, the condition is pain, fever, and/or an inflammatory-related disease or disorder. In another aspect, where the one or more additional lipophilic active agents within the compositions and methods of the invention is a vitamin, particularly vitamin E as described herein, the condition is vitamin E deficiency and/or a vitamin E related disease or disorder.

In a further aspect, a method of enhancing the bioavailability of a lipophilic active agent is provided, comprising heating any of the compositions disclosed herein to a temperature that is greater than or equal to human body temperature. In some aspects, oral administration of any of the compositions disclosed herein to a subject in need thereof results in a heating of the compositions to a temperature that is equal to human body temperature.

Other compositions, methods, features, and advantages of the invention will be or will become apparent to one with skill in the art upon examination of the following figures and detailed description. It is intended that all such additional compositions, methods, features, and advantages be included within this description, be within the scope of the invention, and be protected by the accompanying claims.

### DETAILED DESCRIPTION

The presently disclosed subject matter now will be described more fully hereinafter. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

### I. Compositions

In one aspect, a lipophilic active agent infused food product is provided, comprising: (a) a therapeutically effective amount of a phosphodiesterase type 5 (PDE5 inhibitor), alone or in combination with one or more additional lipophilic active agents; (b) a bioavailability enhancing agent, wherein the bioavailability enhancing agent enhances the bioavailability of the lipophilic active agent(s); and (c) a food product. In another aspect, the lipophilic active agent infused food product is obtainable by the steps of: (i) contacting the food product with an oil comprising the PDE5 inhibitor, alone or in combination with a one or more additional lipophilic active agents, and the bioavailability enhancing agent; and (ii) dehydrating the food product; thereby producing the lipophilic active agent infused food product. In a further aspect, step (i) comprises saturating the food product in the oil comprising the PDE5 inhibitor, alone or in combination with a one or more additional lipophilic active agents, and the bioavailability enhancing agent. In another aspect, the lipophilic active agent infused food product further comprises: (d) a starch. In another aspect, step (ii) comprises contacting the food product with the starch.

In some aspects, the food product is selected from the group consisting of tea leaves, coffee beans, cocoa powder, meats, fish, fruits, vegetables, dairy products, legumes, pastas, breads, grains, seeds, nuts, spices, and herbs. In yet another aspect, the lipophilic active agent infused food product further comprises a flavoring agent, particularly wherein the flavoring agent is selected from the group consisting of vanilla, vanillin, ethyl vanillin, orange oil, peppermint oil, strawberry, raspberry, and mixtures thereof. In a further aspect, the lipophilic active agent infused food product is lyophilized. In a further aspect, wherein the lipophilic active agent infused food product is tea leaves, the process further comprises packaging the tea leaves in tea bags.

In another aspect, a lipophilic active agent infused beverage product is provided that is obtainable by the steps of: (i) providing lipophilic active agent infused tea leaves, coffee beans, or cocoa powder as described herein; and (ii) steeping the lipophilic active agent infused tea leaves, coffee beans, or cocoa powder in a liquid; thereby producing the lipophilic active agent infused beverage product.

A. PDE5 Inhibitors

Phosphodiesterase type 5 inhibitors (PDE5 inhibitors) block the degradative action of cGMP-specific phosphodiesterase type 5 (PDE5) on cyclic GMP in the smooth muscle cells lining the blood vessels supplying the corpus cavernosum of the penis. These drugs, including vardenafil (Levitra®), sildenafil (Viagra®), and tadalafil (Cialis®), are administered orally for the treatment of erectile dysfunction and were the first effective oral treatment available for the condition.

PDE5 inhibitors have also been studied for other clinical use as well, including cardiovascular and heart diseases. For example, because PDE5 is also present in the arterial wall smooth muscle within the lungs, PDE5 inhibitors have also been explored for lung diseases such as pulmonary hypertension and cystic fibrosis. Pulmonary arterial hypertension, a disease characterized by sustained elevations of pulmonary artery pressure, which leads to an increased incidence of failure of the right ventricle of the heart, which in turn can result in the blood vessels in the lungs become overloaded with fluid. Two oral PDE5 inhibitors, sildenafil (Revatio®) and tadalafil (Adcirca®), are approved for the treatment of pulmonary arterial hypertension. PDE5 inhibitors have been found to have activity as both a corrector and potentiator of CFTR protein abnormalities in animal models of cystic fibrosis disease (Lubamba et al., Am. J. Respir. Crit. Care Med. (2008) 177:506-515, Lubamba et al., J. Cystic Fibrosis (2012) 11:266-273). Sildenafil has also been studied as a potential anti-inflammatory treatment for cystic fibrosis. Oral PDE5 inhibitors have also been reported to have anti-remodeling properties and to improve cardiac inotropism, independent of afterload changes, with a good safety profile (Giannetta et al., BMC Medicine (2014) 12:185). However, oral administration of PDE5 inhibitors results in poor and variable bioavailability and also extensive metabolism in the liver (Sandqvist et al., Eur. J. Clin. Pharmacol. (2013) 69:197-207; Mehrotra, Intl. J. Impotence Res. (2007) 19:253-264). If oral doses are increased beyond certain levels, the incidence of systemic side effects increase which prevents the acceptable use of these drugs. (Levitra EMEA Scientific Discussion Document, 2005).

Accordingly, in other aspects, within the compositions and methods of the present invention, the PDE5 inhibitor may include, but is not limited to, avanafil, lodenafil, mirodenafil, sildenafil (or analogs thereof, for example, actetildenafil, hydroxyacetildenafil, or dimethyl-sildenafil), tadalafil, vardenafil, udenafil, acetildenafil, or thiome-thisosildenafil. The structures of these compounds are respectively shown below:

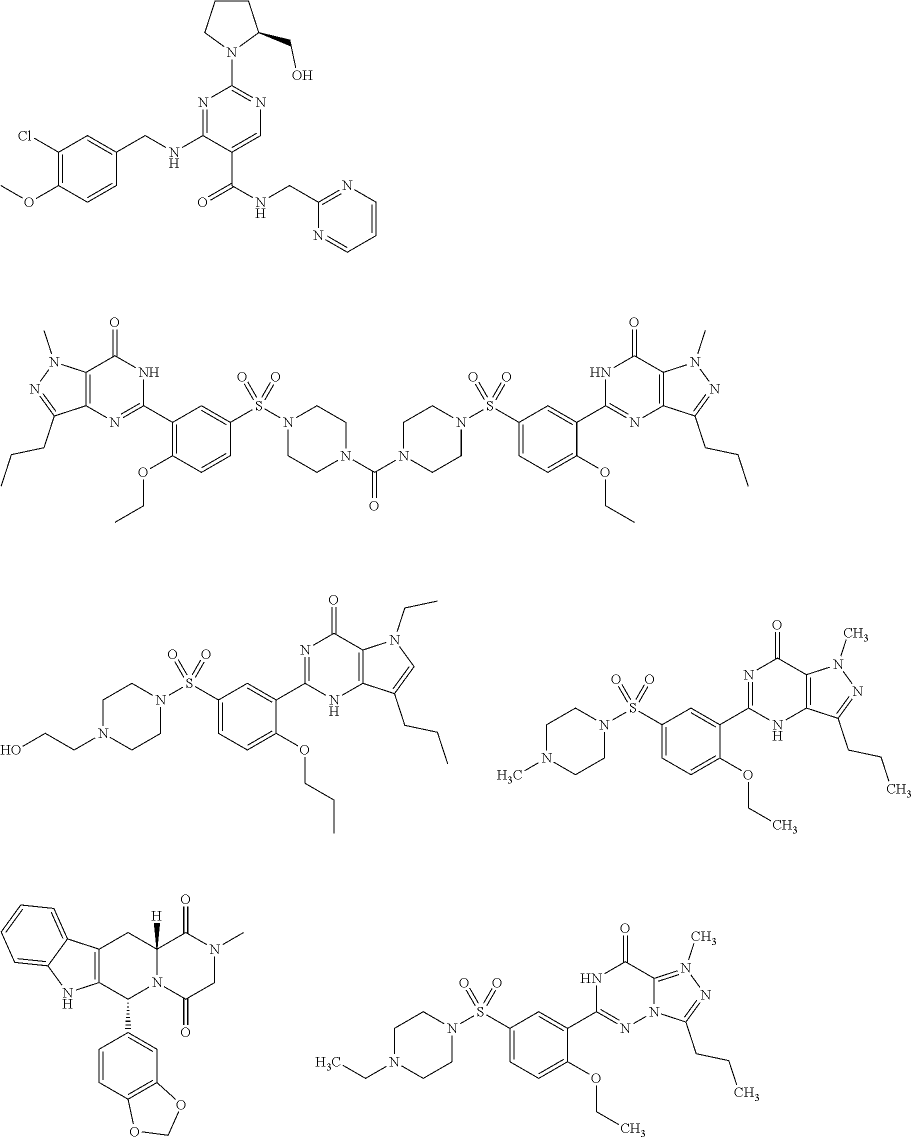

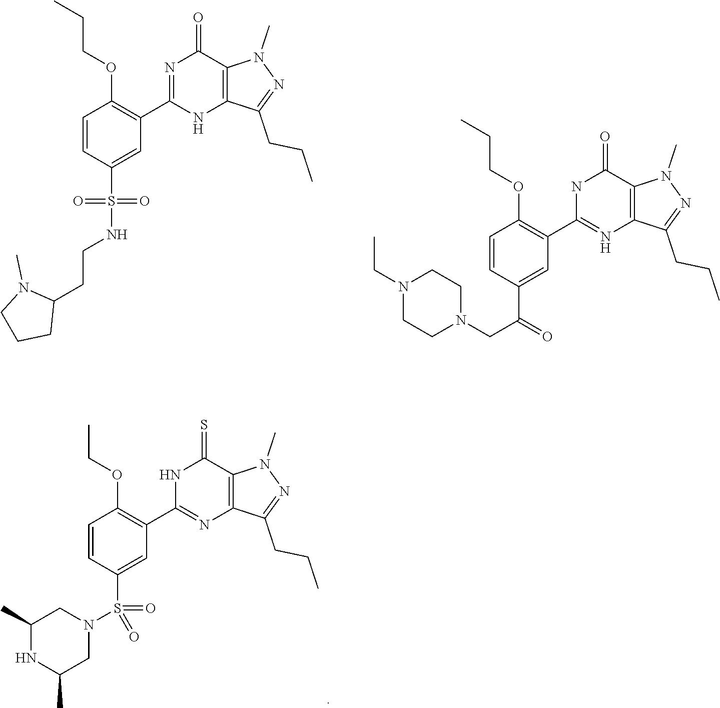 [SMILES: CCCOc1ccc(S(=O)(=O)NCCC2CCCN2C)cc1-c1nc(=O)c2c([nH]1)c(CCC)nn2C ; CCCOc1ccc(C(=O)CN2CCN(CC)CC2)cc1-c1nc2c(CCC)nn(C)c2c(=O)[nH]1 ; CCCc1nn(C)c2c(=S)[nH]c(-c3cc(S(=O)(=O)N4C[C@@H](C)N[C@@H](C)C4)ccc3OCC)nc12]

B. Additional Lipophilic Active Agents

In some aspects, within the compositions and methods of the present invention, the lipophilic active agent is selected from the group consisting of a cannabinoid, nicotine, a non-steroidal anti-inflammatory drug (NSAID), and a vitamin. In other aspects, the cannabinoid is a nonpsychoactive cannabinoid such as cannabidiol. In other aspects, the NSAID is selected from the group consisting of acetylsalicylic acid, ibuprophen, acetaminophen, diclofenac, indomethacin, and piroxicam. In other aspects, the lipophilic active agent is vitamin E.

i. Cannabinoids

Cannabis sativa L. is one of the most widely used plants for both recreational and medicinal purposes. Over 500 natural constituents have been isolated and identified from C. sativa covering several chemical classes (Ahmed et al. (2008) J. Nat. Prod. 71:536-542; Ahmed et al. (2008) Tetrahedron Lett. 49:6050-6053; ElSohly & Slade (2005) Life Sci. 78:539-548; Radwan et al. (2009) J. Nat. Prod. 72:906-911; Radwan et al. (2008) Planta Medica. 74:267-272; Radwan et al. (2008) J. Nat. Prod. 69:2627-2633; Ross et al. (1995) Zagazig J. Pharm. Sci. 4:1-10; Turner et al. (1980) J. Nat. Prod. 43:169-170). Cannabinoids belong to the chemical class of terpenophenolics, of which at least 85 have been uniquely identified in cannabis (Borgelt et al. (2013) Pharmacotherapy 33:195-209).

Cannabinoids are ligands to cannabinoid receptors (CB₁, CB₂) found in the human body (Pertwee (1997) Pharmacol. Ther. 74:129-180). The cannabinoids are usually divided into the following groups: classical cannabinoids; non-classical cannabinoids; aminoalkylindole-derivatives; and eicosanoids (Pertwee (1997) Pharmacol. Ther. 74:129-180). Classical cannabinoids are those that have been isolated from C. sativa L. or their synthetic analogs. Non-classical cannabinoids are bi- or tri-cyclic analogs of tetrahydrocannabinol (THC) (without the pyran ring). Aminoalkylindoles and eicosanoids are substantially different in structure compared to classical and non-classical cannabinoids. The most common natural plant cannabinoids (phytocannabinoids) are cannabidiol (CBD), cannabigerol (CBG), cannabichromene (CBC), and cannabinol (CBN). The most psychoactive cannabinoid is Δ⁹-THC.

In recent years, marijuana and its components have been reported in scientific literature to counter the symptoms of a broad range of conditions including but not limited to multiple sclerosis and other forms of muscular spasm; movement disorders; pain, including migraine headache; glaucoma; asthma; inflammation; insomnia; and high blood pressure. There may also be utility for cannabinoids as anxiolytics, anti-convulsives, anti-depressants, anti-psychotics, anti-cancer agents, as well as appetite stimulants. Pharmacological and toxicological studies of cannabinoids have largely been focused on a synthetic analog of Δ⁹-THC (commercially available under the generic name Dronabinol). In 1985, Dronabinol was approved by the FDA for the treatment of chemotherapy associated nausea and vomiting, and later for AIDS-associated wasting and anorexia.

Therapeutic use of cannabinoids has been hampered by the psychoactive properties of some compounds (e.g., Dronabinol) as well as their low bioavailability when administered orally. Bioavailability refers to the extent and rate at which the active moiety (drug or metabolite) enters systemic circulation, thereby accessing the site of action. The low bioavailability of orally ingested cannabinoids (from about 6% to 20%; Adams & Martin (1996) Addiction 91: 1585-614; Agurell et al. (1986) Pharmacol. Rev. 38: 21-43; Grotenhermen (2003) Clin. Pharmacokinet. 42: 327-60) has been attributed to their poor dissolution properties and extensive first pass metabolism.

Cannabinoids are a heteromorphic group of chemicals which directly or indirectly activate the body's cannabinoid receptors. There are three main types of cannabinoids: herbal cannabinoids that occur uniquely in the cannabis plant, synthetic cannabinoids that are manufactured, and endogenous cannabinoids that are produced in vivo. Herbal cannabinoids are nearly insoluble in water but soluble in lipids, alcohol, and non-polar organic solvents. These natural cannabinoids are concentrated in a viscous resin that is produced in glandular structures known as trichomes. In addition to cannabinoids, the resin is rich in terpenes, which are largely responsible for the odor of the cannabis plant.

The identification of Δ⁹-tetrahydrocannabinol (THC) as a major psychoactive drug and its chemical synthesis in 1964 opened a new era of synthetic cannabinoids as pharmacological agents. Cannabinoid research has increased tremendously in recent years since the discovery of cannabinoid receptors and the endogenous ligands for these receptors. The receptors include CB1, predominantly expressed in the brain, and CB2, primarily found on the cells of the immune system. Cannabinoid receptors belong to a superfamily of G-protein-coupled receptors. They are single polypeptides with seven transmembrane α-helices, and have an extracellular, glycosylated N-terminus and intracellular C-terminus. Both CB1 and CB2 cannabinoid receptors are linked to G1/0-proteins. In addition to these receptors, endogenous ligands for these receptors capable of mimicking the pharmacological actions of THC have also been discovered. Such ligands were designated endocannabinoids and included anandamide and 2-arachidonoyl glycerol (2-AG). Anandamide is produced in the brain and peripheral immune tissues such as the spleen.

Unlike THC, which exerts its action by binding to CB1 and CB2, cannabidiol does not bind to these receptors and hence has no psychotropic activity. Instead, cannabidiol indirectly stimulates endogenous cannabinoid signaling by suppressing the enzyme that breaks down anandamide (fatty acid amide hydroxylase, “FAAH”). Cannabidiol also stimulates the release of 2-AG. Cannabidiol has been reported to have immunomodulating and anti-inflammatory properties, to exhibit anticonvulsive, anti-anxiety, and antipsychotic activity, and to function as an efficient neuroprotective antioxidant.

Cannabinoids in cannabis are often inhaled via smoking, but may also be ingested. Smoked or inhaled cannabinoids have reported bioavailabilities ranging from 2-56%, with an average of about 30% (Huestis (2007) Chem. Biodivers. 4:1770-1804; McGilveray (2005) Pain Res. Manag. 10 Suppl. A:15A-22A). This variability is mainly due to differences in smoking dynamics. Cannabinoids that are absorbed through the mucous membranes in the mouth (buccomucosal application) have bioavailabilities of around 13% (Karschner et al. (2011) Clin. Chem. 57:66-75). By contrast, when cannabinoids are ingested, bioavailability is typically reduced to about 6% (Karschner et al. (2011) Clin. Chem. 57:66-75).

Accordingly, in other aspects, within the compositions and methods of the present invention, the lipophilic active agent is a cannabinoid.

In particular aspects, at least one cannabinoid within the compositions and methods of the present invention is selected from the group consisting of:

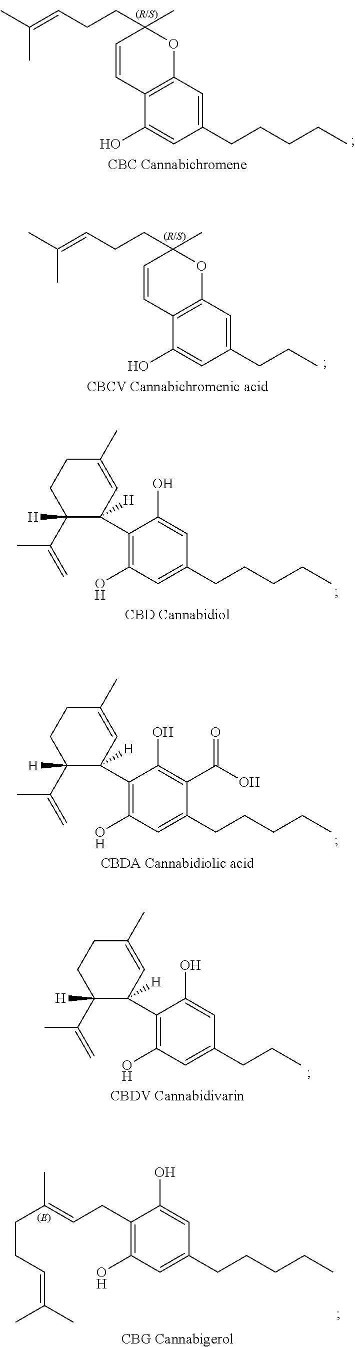

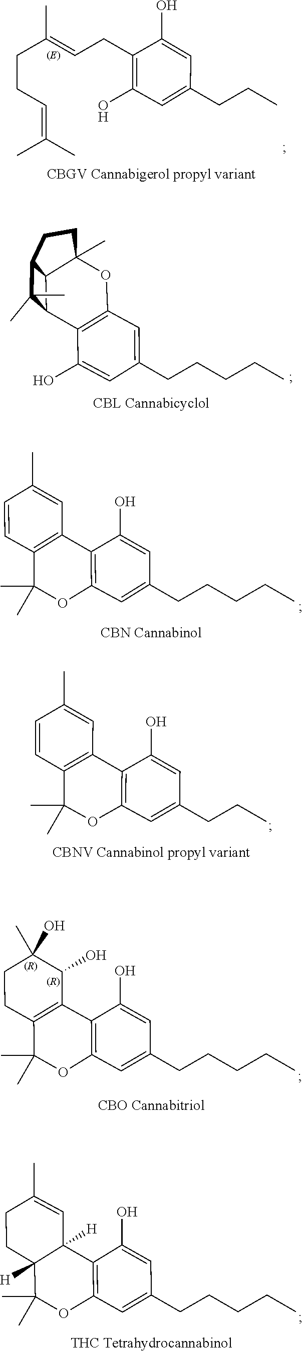

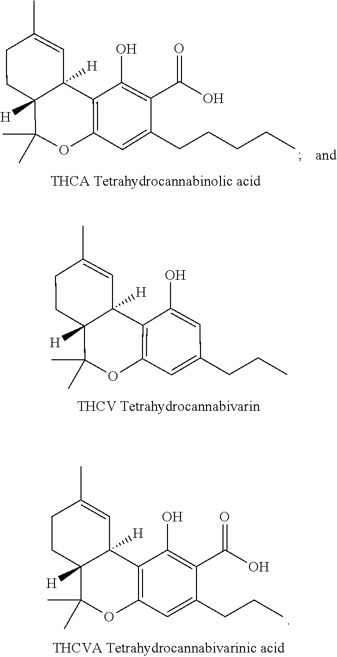

In particular aspects, at least one cannabinoid within the compositions and methods of the present invention is a non-psychoactive cannabinoid such as cannabidiol. In some particularly disclosed aspects, the cannabinoid is selected from the group consisting of:

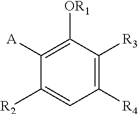

where A is aryl, and particularly

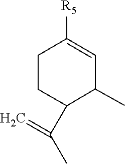

but not a pinene such as:

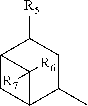

and the R₁-R₅ groups are each independently selected from the groups of hydrogen, lower substituted or unsubstituted alkyl, substituted or unsubstituted carboxyl, substituted or unsubstituted alkoxy, substituted or unsubstituted alcohol, and substituted or unsubstituted ethers, and R₆-R₇ are H or methyl. In particular aspects, there are no nitrogens in the rings, and/or no amino substitutions on the rings.

In other aspects, the cannabinoid is selected from the group consisting of:

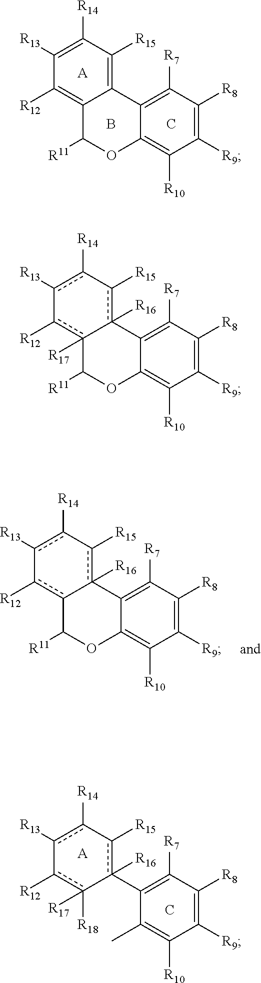

where there can be 0 to 3 double bonds on the A ring, as indicated by the optional double bonds indicated by dashed lines on the A ring. The C ring is aromatic, and the B ring can be a pyran. Particular aspects are dibenzo pyrans and cyclohexenyl benzenediols. Particular aspects of the cannabinoids of the present invention may also be highly lipid soluble, and in particular aspects can be dissolved in an aqueous solution only sparingly (for example 10 mg/ml or less). The octanol/water partition ratio at neutral pH in useful aspects is 5000 or greater, for example 6000 or greater. This high lipid solubility enhances penetration of the drug into the central nervous system (CNS), as reflected by its volume of distribution (V_{d}) of 1.5 L/kg or more, for example 3.5 L/kg, 7 L/kg, or ideally 10 L/kg or more, for example at least 20 L/kg. Particular aspects may also be highly water soluble derivatives that are able to penetrate the CNS, for example carboxyl derivatives.

R₇₋₁₈ are independently selected from the group of H, substituted or unsubstituted alkyl, especially lower alkyl, for example unsubstituted C₁-C₃ alkyl, hydroxyl, alkoxy, especially lower alkoxy such as methoxy or ethoxy, substituted or unsubstituted alcohol, and unsubstituted or substituted carboxyl, for example COOH or COCH₃. In other aspects R₇₋₁₈ can also be substituted or unsubstituted amino, and halogen.

In particular aspects, at least one cannabinoid within the compositions and methods of the present invention is a non-psychoactive cannabinoid, meaning that the cannabinoid has substantially no psychoactive activity mediated by the cannabinoid receptor (for example an IC₅₀ at the cannabinoid receptor of greater than or equal to 300 nM, for example greater than 1 μM and a Kᵢ greater than 250 nM, especially 500-1000 nM, for example greater than 1000 nM).

In other particular aspects, the cannabinoids within the compositions and methods of the present invention are selected from the group consisting of:

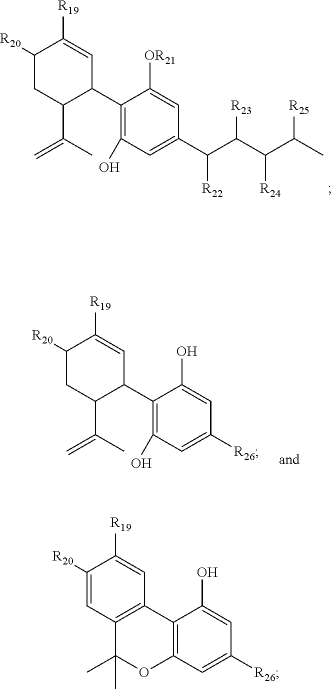

where R₁₉ is substituted or unsubstituted alkyl, such as lower alkyl (for example methyl), lower alcohol (such as methyl alcohol) or carboxyl (such as carboxylic acid) and oxygen (as in ═O); R₂₀ is hydrogen or hydroxy; R₂₁ is hydrogen, hydroxy, or methoxy; R₂₂ is hydrogen or hydroxy; R₂₃ is hydrogen or hydroxy; R₂₄ is hydrogen or hydroxy; R₂₅ is hydrogen or hydroxy; and R₂₆ is substituted or unsubstituted alkyl (for example n-methyl alkyl), substituted or unsubstituted alcohol, or substituted or unsubstituted carboxy.

In other particular aspects, the cannabinoids within the compositions and methods of the present invention are selected from the group consisting of:

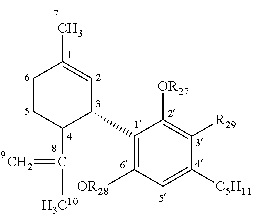

wherein numbering conventions for each of the ring positions are shown, and R₂₇, R₂₈ and R₂₉ are independently selected from the group consisting of H, unsubstituted lower alkyl such as CH₃, and carboxyl such as COCH₃. Particular examples of nonpsychoactive cannabinoids that fall within this definition are cannabidiol and

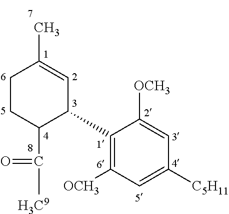

and other structural analogs of cannabidiol.

In other particular aspects, the cannabinoids within the compositions and methods of the present invention are selected from the group consisting of:

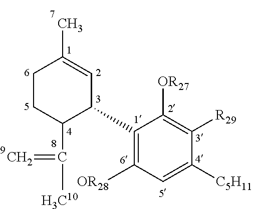

wherein R₂₇, R₂₈ and R₂₉ are independently selected from the group consisting of H, lower alkyl such as CH₃, and carboxyl such as COCH₃, and particularly wherein:
a) R₂₇═R₂₈═R₂₉═H
b) R₂₇═R₂₉═H; R₂₈═CH₃
c) R₂₇═R₂₈═CH₃; R₂₉═H
d) R₂₇═R₂₈═COCH₃; R₂₉═H
e) R₂₇═H; R₂₈═R₂₉═COCH₃
   When R₂₇═R₂₈═R₂₉═H, then the compound is cannabidiol (CBD). When R₂₇═R₂₉═H and R₂₈═CH₃, the compound is CBD monomethyl ether. When R₂₇═R₂₈═CH₃ and R₂₉═H, the compound is CBD dimethyl ether. When R₂₇═R₂₈═COCH₃ and R₂₉═H, the compound is CBD diacetate. When R₂₇═H and R₂₈═R₂₉═COCH₃, the compound is CBD monoacetate.

In yet another aspect, cannabinoid infused tea leaves are packaged in tea bags, wherein each tea bag comprises 1 to 3 grams of tea leaves (dry weight), 0.10 to 1.0 grams of dry milk, and 10 to 25 mg of cannabinoid oil. In still another aspect, the cannabinoid infused tea leaves are packaged in tea bags, wherein each tea bag comprises 1.5 to 12 grams of tea leaves (dry weight), 0.10 to 6.0 grams of dry milk, 10 to 25 mg of hemp oil, and 1.0 to 12.0 grams of cannabis leaves.

ii. Nicotine

More than 99% of all nicotine that is consumed worldwide is delivered through smoking cigarettes. Approximately 6,000,000 deaths per year, worldwide, are attributed primarily to the delivery of nicotine through the act of smoking according to the Centers for Disease Control and Prevention, which also estimates that over $170 billion per year is spent just in the U.S. on direct medical care costs for adult smokers. In any twelve month period, 69% of U.S. adult smokers want to quit smoking and 43% of U.S. adult smokers have attempted to quit.

Worldwide, retail cigarette sales were worth $722 billion in 2013, with over 5.7 trillion cigarettes sold to more than 1 billion smokers.

The delivery of nicotine to satisfy current demand via the compositions and methods of the present invention (i.e., in common food groups), will alleviate the consumer demand for cigarettes. Since most of the adverse health outcomes of nicotine consumption are associated with the delivery method and only to a lesser degree to the actual ingestion of nicotine, a vast positive community health outcome can be achieved through the reduction in smoking cigarettes.

Accordingly, in other aspects, within the compositions and methods of the present invention, the lipophilic active agent is nicotine.

iii. Non-Steroidal Anti-inflammatory Drugs (NSAIDs)

NSAIDs are the second-largest category of pain management treatment options in the world. The global pain management market was estimated at $22 billion in 2011, with $5.4 billion of this market being served by NSAID's. The U.S. makes up over one-half of the global market. The opioids market (such as morphine) form the largest single pain management sector but are known to be associated with serious dependence and tolerance issues.

Although NSAIDs are generally a safe and effective treatment method for pain, they have been associated with a number of gastrointestinal problems including dyspepsia and gastric bleeding.

Delivery of NSAIDs through the compositions and methods of the present invention will provide the beneficial properties of pain relief with lessened negative gastrointestinal effects, and also deliver lower dosages of active ingredients with similar pain management outcomes as current pill forms at higher dosages.

Accordingly, in other aspects, within the compositions and methods of the present invention, the lipophilic active agent is an NSAID, particularly wherein the NSAID is selected from the group consisting of acetylsalicylic acid, ibuprophen, acetaminophen, diclofenac, indomethacin, and piroxicam.

iv. Vitamins

The global vitamin and supplement market is worth $68 billion according to Euromonitor. The category is both broad and deep, comprised of many popular and some lesser known substances. Vitamins in general are thought to be an $8.5 billion annual market in the U.S. The U.S. is the largest single national market in the world, and China and Japan are the 2^{nd} and 3^{rd} largest vitamin markets.

Vitamin E is fat soluble and can be incorporated into cell membranes which can protect them from oxidative damage. Global consumption of natural source vitamin E was 10,900 metric tons in 2013 worth $611.9 million.

Delivery of fat soluble vitamins through the compositions and methods of the present invention will result in less waste and lower dosages required than current pill forms. In addition, ingestion of pills is an unpleasant experience for many people so vitamin delivery through common food groups will vastly expand demand and use.

Accordingly, in other aspects, within the compositions and methods of the present invention, the lipophilic active agent is a vitamin, particularly wherein the vitamin is vitamin E.

C. Edible Oils or Fats

An edible oil is defined herein as an oil that is capable of undergoing de-esterification or hydrolysis in the presence of pancreatic lipase in vivo under normal physiological conditions. Specifically, digestible oils may be complete glycerol triesters of medium chain (C₇-C₁₃) or long chain (C₁₄-C₂₂) fatty acids with low molecular weight (up to C₆) mono-, di- or polyhydric alcohols. Some examples of digestible oils for use in this invention thus include: vegetable, nut, or seed oils (such as coconut oil, peanut oil, soybean oil, safflower seed oil, corn oil, olive oil, castor oil, cottonseed oil, arachis oil, sunflower seed oil, coconut oil, palm oil, rapeseed oil, evening primrose oil, grape seed oil, wheat germ oil, sesame oil, avocado oil, almond, borage, peppermint and apricot kernel oils) and animal oils (such as fish liver oil, shark oil and mink oil).

D. Starches

In some aspects, within the compositions and methods of the present invention, the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

E. Bioavailability Enhancing Agents

Bioavailability refers to the extent and rate at which the active moiety (drug or metabolite) enters systemic circulation, thereby accessing the site of action. Bioavailability for a given formulation provides an estimate of the relative fraction of the orally administered dose that is absorbed into the systemic circulation. Low bioavailability is most common with oral dosage forms of poorly water-soluble, slowly absorbed drugs. Insufficient time for absorption in the gastrointestinal tract is a common cause of low bioavailability. If the drug does not dissolve readily or cannot penetrate the epithelial membrane (e.g., if it is highly ionized and polar), time at the absorption site may be insufficient. Orally administered drugs must pass through the intestinal wall and then the portal circulation to the liver, both of which are common sites of first-pass metabolism (metabolism that occurs before a drug reaches systemic circulation). Thus, many drugs may be metabolized before adequate plasma concentrations are reached.

Bioavailability is usually assessed by determining the area under the plasma concentration-time curve (AUC). AUC is directly proportional to the total amount of unchanged drug that reaches systemic circulation. Plasma drug concentration increases with extent of absorption; the maximum (peak) plasma concentration is reached when drug elimination rate equals absorption rate. Peak time is the most widely used general index of absorption rate; the slower the absorption, the later the peak time.

The bioavailability of some drugs is increased when co-administered with food, particularly agents such as cannabinoids that are Class II drugs under the Biopharmaceutical Drug Classification System (Kelepu et al. (2013) Acta Pharmaceutica Sinica B 3:361-372; Amidon et al. (1995) Pharm. Res. 12:413-420; Charman et al. (1997) J. Pharm. Sci. 86:269-282; Winstanley et al. (1989) Br. J. Clin. Pharmacol. 28:621-628). It is the lipid component of the food that plays a key role in the absorption of lipophilic drugs and that leads to enhanced oral bioavailability (Hunt & Knox (1968) J. Physiol. 194:327-336; Kelepu et al. (2013) Acta Pharmaceutica Sinica B 3:361-372). This has been attributed to the ability of a high fat meal to stimulate biliary and pancreatic secretions, to decrease metabolism and efflux activity, to increase intestinal wall permeability, and to a prolongation of gastrointestinal tract (GIT) residence time and transport via the lymphatic system (Wagnera et al. (2001) Adv. Drug Del. Rev. 50:S13-31; Kelepu et al. (2013) Acta Pharmaceutica Sinica B 3:361-372). High fat meals also elevate triglyceride-rich lipoproteins that associate with drug molecules and enhance intestinal lymphatic transport, which leads to changes in drug disposition and changes the kinetics of the pharmacological actions of poorly soluble drugs (Gershkovich et al. (2007) Eur. J. Pharm. Sci. 32:24-32; Kelepu et al. (2013) Acta Pharmaceutica Sinica B 3:361-372). However, co-administration of food with lipophilic drugs requires close control and/or monitoring of food intake when dosing such drugs, and can also be subject to problems with patient compliance (Kelepu et al. (2013) Acta Pharmaceutica Sinica B 3:361-372).

In some aspects, within the compositions and methods of the present invention, the bioavailability enhancing agent is an edible oil or fat, a protective colloid, or both a protective colloid and an edible oil or fat. In another aspect, the bioavailability enhancing agent is also a lipophilic active agent taste masking agent. In another particular aspect, where the bioavailability enhancing agent is both a protective colloid, an edible oil or fat, and a lipophilic active agent taste masking agent, the bioavailability enhancing agent is nonfat dry milk. In a further aspect, the bioavailability enhancing agent is substantially free of omega-6 fatty acids. In other aspects, the bioavailability of the lipophilic active agent in a subject is at least about 1.5 times, 2 times, 5 times, or 10 times greater than the bioavailability of the lipophilic active agent in the subject in the absence of the bioavailability enhancing agent. In a further aspect, the bioavailability of the lipophilic active agent in a subject is greater than 20%.

Examples of protective colloids include polypeptides (such as gelatin, casein, and caseinate), polysaccharides (such as starch, dextrin, dextran, pectin, and gum arabic), as well as whole milk, skimmed milk, milk powder or mixtures of these. However, it is also possible to use polyvinyl alcohol, vinyl polymers, for example polyvinylpyrrolidone, (meth)acrylic acid polymers and copolymers, methylcellulose, carboxymethylcellulose, hydroxypropylcellulose and alginates. For further details, reference may be made to R. A. Morton, Fast Soluble Vitamins, Intern. Encyclopedia of Food and Nutrition, Vol. 9, Pergamon Press 1970, pages 128-131.

Oral administration constitutes the preferred route of administration for a majority of drugs. However, drugs that have an undesirable or bitter taste leads to lack of patient compliance in the case of orally administered dosage forms. In such cases, taste masking is an essential tool to improve patient compliance. Because lipophilic active agents (e.g., cannabinoids such as cannabidiol) have an undesirable taste profile, in order to improve compliance, the presently disclosed compositions also comprise one or more lipophilic active agent taste masking agents. Examples of lipophilic active agent taste-masking agents include dry milk as described above, as well as menthol, sweeteners, sodium bicarbonate, ion-exchange resins, cyclodextrin inclusion compounds, adsorbates, and the like.

In a further aspect, the bioavailability enhancing agent is substantially free of omega-6 fatty acids.

In other aspects, the bioavailability of the lipophilic active agent in a subject is at least about 1.5 times, 2 times, 2.5 times, 3 times, 3.5 times, 4 times, 4.5 times, 5 times, 5.5 times, 6 times, 6.5 times, 7 times, 7.5 times, 8 times, 8.5 times, 9 times, 9.5 times, or 10 times greater than the bioavailability of the lipophilic active agent in the subject in the absence of the bioavailability enhancing agent.

In a further aspect, the bioavailability of the lipophilic active agent in a subject is greater than 20% or at least about 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or greater.

Assays and methods for measuring lipophilic active agent bioavailability are well known in the art (see, e.g., Rocci & Jusko (1983) Comput. Programs Biomed. 16:203-215; Shargel & Yu (1999) Applied biopharmaceutics & pharmacokinetics (4th ed.). New York: McGraw-Hill; Hu & Li (2011) Oral Bioavailability: Basic Principles, Advanced Concepts, and Applications, John Wiley & Sons Ltd.; Karschner et al. (2011) Clinical Chemistry 57:66-75; Ohlsson et al. (1980) Clin. Pharmacol. Ther. 28:409-416; Ohlsson et al. (1982) Biomed. Environ. Mass Spectrom. 9:6-10; Ohlsson et al. (1986) Biomed. Environ. Mass Spectrom. 13:77-83; Karschner et al. (2010) Anal. Bioanal. Chem. 397:603-611).

E. Flavoring Agents

In some aspects, within the compositions and methods of the present invention, the flavoring agent is selected from the group consisting of vanilla, vanillin, ethyl vanillin, orange oil, peppermint oil, strawberry, raspberry, and mixtures thereof.

G. Dosages

The active agents of the present invention are effective over a wide dosage range. For example, in treating adult humans, compositions and methods of the present invention comprise dosages of lipophilic active agents from 0.01 mg to 1,000 mg, from 0.5 mg to 500 mg, from 1 mg to 100 mg, from 5 mg to 50 mg, and from 10 mg to 25 mg. Alternatively, in treating adult humans, compositions and methods of the present invention comprise dosages of lipophilic active agents of 0.01 mg, 0.05 mg, 0.1 mg, 0.25 mg, 0.5 mg, 0.75 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1,000 mg.

The oral administration of PDE5 inhibitors such as sildenafil is accompanied by dose-responsive undesirable side effects such as syncope (loss of consciousness), priapism (erection lasting 4 hours or more), and increased cardiac risk (coital coronaries). It is noted these can be brought on in some cases by physiological predisposition, adverse drug interaction or potentiation, or by drug abuse. In particular, hypotension crisis can result from the combination of sildenafil citrate and organic nitrates, causing death in some cases, so its administration to patients who are concurrently using organic nitrates (such as nitroglycerin) in any form is contraindicated. Thus, there is a need and desire for oral administration forms that promote the bioavailability of PDE5 inhibitors such as sildenafil at lower doses while minimizing side effects. Accordingly, the compositions of the present invention allow for the avoidance of undesirable side effects of PDE5 inhibitors such as sildenafil by providing the same efficacy at lower dosage levels due to improved bioavailability and absorption, alone or in combination with other lipophilic active agents such as cannabinoids, nicotine, non-steroidal anti-inflammatory drugs (NSAIDs), and vitamins. In instances where agents such as cannabinoids like Cannabidiol or NSAIDs such as acetylsalicylic acid are used, such agents may offer complementary vasodilator effects that are beneficial together with the PDE5 inhibitor.

H. Lyophilization

Lyophilization, also known as freeze-drying, is a process whereby water is sublimed from a composition after it is frozen. The frozen solution is then typically subjected to a primary drying step in which the temperature is gradually raised under vacuum in a drying chamber to remove most of the water, and then to a secondary drying step typically at a higher temperature than employed in the primary drying step to remove the residual moisture in the lyophilized composition. The lyophilized composition is then appropriately sealed and stored for later use. Tang et al. (2004) Pharmaceutical Research 21:191-200 describes the scientific principles pertaining to freeze drying and guidelines for designing suitable freeze drying processes. Further description of freeze drying is found in Remington (2006) The Science and Practice of Pharmacy, 21^{st} edition, Lippincott Williams & Wilkins, pp. 828-831.

I. Pharmaceutical Compositions

In another aspect, a pharmaceutical composition is provided, comprising (a) a therapeutically effective amount of a PDE5 inhibitor, alone or in combination with one or more additional lipophilic active agents; and (b) a bioavailability enhancing agent, wherein the bioavailability enhancing agent enhances the bioavailability of the lipophilic active agent(s). In another aspect, the pharmaceutical composition further comprises: (c) a starch. In a further aspect, the pharmaceutical composition is formulated for oral administration. In some aspects, the pharmaceutical composition formulated for oral administration is formulated as a tablet, pill, capsule, liquid, gel, syrup, or slurry.

The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery. In a particular embodiment, the pharmaceutical composition is formulated for oral administration.

Active agents can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers, such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof, such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler, such as lactose, binders, such as starches, and/or lubricants, such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added. In some embodiments, the pharmaceutical composition is formulated for oral administration.

### II. PROCESSES

In another aspect, the lipophilic active agent infused food product is obtainable by the steps of: (i) contacting the food product with an oil comprising the PDE5 inhibitor, alone or in combination with a one or more additional lipophilic active agents, and the bioavailability enhancing agent; and (ii) dehydrating the food product; thereby producing the lipophilic active agent infused food product. In a further aspect, step (i) comprises saturating the food product in the oil comprising the PDE5 inhibitor, alone or in combination with a one or more additional lipophilic active agents, and the bioavailability enhancing agent. In another aspect, step (ii) comprises contacting the food product with the starch. In another aspect, step (i) comprises contacting the food product with a flavoring agent, particularly wherein the flavoring agent is selected from the group consisting of vanilla, vanillin, ethyl vanillin, orange oil, peppermint oil, strawberry, raspberry, and mixtures thereof. In another aspect, the process further comprises a step of lyophilizing the lipophilic active agent infused food product.

In a further aspect, where the lipophilic active agent infused food product is tea leaves, coffee beans, or cocoa powder, the process further comprises packaging the lipophilic active agent infused tea leaves, coffee beans, or cocoa powder in single or multiple serve delivery devices, such as tea bags, water permeable membranes, pre-packaged beverage pods such as K-CUP® packs manufactured and sold by Keurig Inc. of Wakefield, Mass., and the like. Examples include, but are not limited to, such delivery devices and related systems as described in U.S. Pat. Nos. 3,450,024, 5,325,765; 5,840,189; and 6,606,938.

In another aspect, a process for making a lipophilic active agent infused beverage product is provided comprising making lipophilic active agent infused tea leaves, coffee beans, or cocoa powder according to any of the processes described herein; further comprising the step of steeping the lipophilic active agent infused tea leaves, coffee beans, or cocoa powder in a liquid, thereby producing the lipophilic active agent infused beverage product.

### Methods of Treatment

In a further aspect, a method of treating one or more conditions is provided, comprising administering any of the compositions disclosed herein to a subject in need thereof.

In one aspect, the condition is a PDE5-related disorder, particularly erectile dysfunction.

In one aspect, where the one or more additional lipophilic active agents within the compositions and methods of the invention is a cannabinoid, the one or more conditions is selected from the group consisting of cardiac diseases such as heart disease, ischemic infarcts, and cardiometabolic disorders; neurological diseases such as Alzheimer's disease, Parkinson's disease, schizophrenia, and Human Immunodeficiency Virus (HIV) dementia; obesity; metabolic disorders such as insulin related deficiencies and lipid profiles, hepatic diseases, diabetes, and appetite disorders; cancer chemotherapy; benign prostatic hypertrophy; irritable bowel syndrome; biliary diseases; ovarian disorders; marijuana abuse; and alcohol, opioid, nicotine, or cocaine addiction.

In another aspect, where the one or more lipophilic active agents within the compositions and methods of the invention is nicotine, the one or more conditions is a nicotine-related disorder such as tobacco dependence/addiction, Parkinson's disease, ulcerative colitis, Alzheimer's disease, schizophrenia, Attention Deficit Hyperactivity Disorder (ADHD), Tourette's syndrome, ulcerous colitis, and post-smoking-cessation weight control.

In another aspect, where the one or more additional lipophilic active agents within the compositions and methods of the invention is an NSAID as described herein, the one or more conditions is pain, fever, and/or an inflammatory-related disease or disorder, including but not limited to asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

In another aspect, where the one or more lipophilic active agents within the compositions and methods of the invention is a vitamin, the one or more conditions is a vitamin deficiency or condition associated with the lipophilic vitamin. In a particular aspect, where the vitamin is vitamin E as described herein, the condition is vitamin E deficiency and/or a vitamin E related disease or disorder such as ataxia associated with vitamin E deficiency.

In a further aspect, a method of enhancing the bioavailability of a lipophilic active agent is provided, comprising heating any of the compositions disclosed herein to a temperature that is greater than or equal to human body temperature. In some aspects, oral administration of any of the compositions disclosed herein to a subject in need thereof results in a heating of the compositions to a temperature that is equal to human body temperature.

In another aspect, a method of administering any of the lipophilic active agents described herein to a subject is provided, comprising oral administration of any of the compositions of the present invention. Such administration may be for any purpose, including overall health and wellness, mental acuity, alertness, recreation, and the like.

As used herein, the term “subject” treated by the presently disclosed methods in their many aspects is desirably a human subject, although it is to be understood that the methods described herein are effective with respect to all vertebrate species, which are intended to be included in the term “subject.” Accordingly, a “subject” can include a human subject for medical purposes, such as for the diagnosis or treatment of an existing disease, disorder, condition or the prophylactic diagnosis or treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some aspects, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Actual dosage levels of the active ingredients in the presently disclosed compositions can be varied so as to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular composition employed, the route of administration, the time of administration, the rate of excretion of the particular composition being employed, the duration of the treatment, other drugs, and/or materials used in combination with the particular composition employed, the age, sex, weight, condition, general health and prior medical history of the patient being treated, and like factors well known in the medical arts.

A physician having ordinary skill in the art can readily determine and prescribe the effective amount of the presently disclosed composition required. Accordingly, the dosage range for administration may be adjusted by the physician as necessary, as described more fully elsewhere herein.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

1.-36. (canceled)

37. A food product comprising:
(a) a therapeutically effective amount of a phosphodiesterase type 5 (PDE5 inhibitor) in combination with one or more additional lipophilic active agents;
(b) a bioavailability enhancing agent, wherein the bioavailability enhancing agent enhances the bioavailability of the PDE5 inhibitor and the one or more additional lipophilic active agents; and
(c) a food product, wherein the food product is selected from the group consisting of tea leaves, coffee beans, cocoa powder, meats, fish, fruits, vegetables, dairy products, legumes, pastas, breads, grains, seeds, nuts, spices, and herbs.

38. The food product according to claim 37, further comprising:
(d) a starch.

39. The food product according to claim 38, wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

40. The food product according to claim 37, wherein the PDE5 inhibitor is selected form the group consisting of avanafil, lodenafil, mirodenafil, sildenafil (or analogs thereof, for example, actetildenafil, hydroxyacetildenafil, or dimethylsildenafil), tadalafil, vardenafil, udenafil, acetildenafil, and thiomethisosildenafil.

41. The food product according to claim 37, wherein the one or more additional lipophilic active agents is a cannabinoid.

42. The food product according to claim 41, wherein the one or more additional lipophilic active agents is a nonpsychoactive cannabinoid.

43. The food product according to claim 41, wherein the one or more additional lipophilic active agents is a psychoactive cannabinoid.

44. The food product according to claim 37, wherein the one or more additional lipophilic active agents is an NSAID selected from the group consisting of acetylsalicylic acid, ibuprophen, acetaminophen, diclofenac, indomethacin, and piroxicam.

45. The food product according to claim 37, wherein the one or more additional lipophilic active agents is vitamin E.

46. The food product according to claim 37, wherein the bioavailability enhancing agent is a protective colloid, an edible oil or fat, and a lipophilic active agent taste masking agent, further wherein the bioavailability enhancing agent is nonfat dry milk.

47. The food product according to claim 46, wherein the bioavailability of the PDE5 inhibitor and the one or more additional lipophilic active agents in a subject is at least about 1.5 times greater than the bioavailability of the lipophilic active agent in the subject in the absence of the bioavailability enhancing agent.

48. The food product according to claim 46, wherein the bioavailability of the PDE5 inhibitor and the one or more additional lipophilic active agents in a subject is greater than 20%.

49. A process for preparing a lipophilic active agent infused food product, comprising:
(i) combining a food product with an oil comprising:
a) a PDE5 inhibitor;
b) one or more additional lipophilic active agents, and
c) a bioavailability enhancing agent;
to form an infused food product; and
(ii) dehydrating the food product;
thereby producing the lipophilic active agent infused food product.

50. The process according to claim 49, wherein step (i) comprises saturating the food product in the oil comprising the PDE5 inhibitor in combination with the one or more additional lipophilic active agents, and the bioavailability enhancing agent.

51. The process according to claim 49, wherein step (ii) comprises contacting the food product with a starch.

52. The process according to claim 51, wherein the starch is selected from the group consisting of tapioca starch, corn starch, potato starch, gelatin, dextrin, cyclodextrin, oxidized starch, starch ester, starch ether, crosslinked starch, alpha starch, octenylsuccinate ester, and processed starch obtained by treating a starch by an acid, heat, or enzyme.

53. The process according to claim 49, wherein the PDE5 inhibitor is selected form the group consisting of avanafil, lodenafil, mirodenafil, sildenafil (or analogs thereof, for example, actetildenafil, hydroxyacetildenafil, or dimethyl sildenafil), tadalafil, vardenafil, udenafil, acetildenafil, and thiomethisosildenafil.

54. The process according to claim 49, wherein the one or more additional lipophilic active agents is a cannabinoid.

55. The process according to claim 49, wherein the lipophilic active agent is infused tea leaves, coffee beans, or cocoa powder.

56. The process according to claim 55, further comprising:
(iii) steeping the lipophilic active agent infused tea leaves, coffee beans, or cocoa powder in a liquid.
